(19) United States
(12) Patent Application Publication
(10) Pub. No.: US 2016/0067526 A1
(43) Pub. Date: Mar. 10, 2016

(54) Method for treating and/or preventing neurodegenerative diseases by using low-intensity pulsed ultrasound (LIPUS)

(71) Applicant: NATIONAL YANG-MING UNIVERSITY, Taipei (TW)

(72) Inventor: Feng-Yi Yang, Taipei (TW)

(21) Appl. No.: 14/663,152

(22) Filed: Mar. 19, 2015

(30) Foreign Application Priority Data
Sep. 4, 2014 (TW) 103130608

(51) Int. Cl. A61N 7/00

(52) U.S. Cl. CPC A61N 7/00; A61N 2007/0026; A61N 2007/0039

(57) ABSTRACT

This present invention discloses a method for treating and/or preventing neurodegenerative diseases by applying low-intensity pulsed ultrasound (LIPUS) stimulation. In addition, this present invention discloses the LIPUS increases neurotrophic factor protein expression, improves cognitive dysfunction and reduces brain damage in neurotoxicity.

### BACKGROUND OF THE INVENTION

1. Technical Field

The present invention relates to a method for neuroprotection by using low-intensity pulsed ultrasound. In more details, the present invention relates to a method for treating and/or preventing neurodegenerative diseases by using low-intensity pulsed ultrasound. Especially, the present invention relates to the modulation of the frequency, intensity, pulse duration, beam pathway, and other acoustic parameters of low-intensity pulsed ultrasound to target and regulate neurotrophic factor protein expression.

2. Related Art

Ultrasound (US) can be transmitted into a target tissue and produce physiological change through thermal or non-thermal effects. Low-intensity pulsed US (LIPUS) has been known to accelerate bone and tissue regeneration following injury (Tempany, et al., Radiology, 226:897-905, 2003; Lu, et al., The American journal of sports medicine, 34:1287-1296, 2006). Previous studies have also indicated that LIPUS has positive effects on axonal regeneration in damaged nerves (Crisci & Ferreira, Ultrasound in medicine & biology, 28: 1335-1341, 2002). Transcranial pulsed US is capable of stimulating intact brain circuitry and promoting levels of brain-derived neurotrophic factor (BDNF), an important regulator of long-term memory. These findings suggest the potential for broad applications in neuroscience, including the enhancement of neurotrophic factor levels via LIPUS, which could have beneficial effects against degenerative brain diseases.

Aluminum (Al) exposure is known to be neurotoxic and can induce cognitive deficiency and dementia. Although the connection between Alzheimer's disease (AD) and Al still exists controversies, experimentally it has been demonstrated that chronic exposure to Al causes neuropathological changes and cognitive impairments which are similar to those of AD. Al accelerates Aβ generation and increases the formation of beta-amyloid (Aβ) oligomers. It has also been reported that chronic aluminum chloride (AlCl₃) administration in rats showed significant increase in the brain acetylcholinesterase (AChE) activity as compared to control rats. At the cellular and molecular levels, AD is characterized by the deficiency of the neurotransmitter acetylcholine, extracellular Aβ deposits, neurofibrillary tangles, and the loss of neurons.

The brain is protected from entry of foreign substances by the blood-brain barrier (BBB), which is a highly specialized brain endothelial structure. However, an intact BBB is also a major obstacle for the treatment of brain disorders with certain drugs because it prevents large molecule neurotherapeutics from entering the brain. In recent years, neurodegenerative diseases, such as AD and Parkinson's disease (PD), have presented some of the greatest public health challenges to the world's aging populations. Various studies have shown, however, that BDNF has great potential for the treatment of AD. Meanwhile, glial cell line-derived neurotrophic factor (GDNF), another neurotrophic factor, has been identified as the most suitable candidate for the treatment of PD. Besides, vascular endothelial growth factor (VEGF) modulates axonal growth and new vessel formation. Furthermore, a growing body of evidence suggests that focused US (FUS)-induced BBB disruption may be a useful tool for delivering such neurotrophic factors directly into the central nervous system, and increased levels of BDNF and GDNF may lead to neuronal regeneration and a strong trophic effect on the dopaminergic system, respectively. On the other hand, exogenous BDNF and GDNF could also have possible side effects such as a pro-epileptic effect and cerebella damage, respectively. Moreover, AD may be aggravated by a breakdown of the BBB in some patients. Microvascular length is reduced in neurodegenerative diseases (such as AD, for example), and the transport of energy substrates across the BBB and the clearance of potential neurotoxins from the brain would be decreased due to these vascular reductions. Recent FDG (18-fludeoxyglucose)-PET (positron emission tomography) imaging studies have demonstrated that individuals with mild cognitive impairment have significantly reduced glucose utilization prior to neurodegeneration. In addition, the protein expression of glucose transporter 1 (GLUT1) in brain capillaries is decreased in AD. These findings suggest that a continuous shortage in metabolic activity at the BBB occurs due to GLUT1 deficiency. Besides, the transcription factor c-Fos is strongly implicated in memory formation and can be used as memory markers (Tsai, et al., PLoS ONE, 6(8) e24001. doi:10.1371/journal.pone.0024001, 2011). Moreover, CREB (cyclic AMP response element-binding protein) may be a universal modulator of processes required for memory formation (Finkbeiner, et al., Neuron, 19: 1031-1047, 1997).

FUS with microbubbles may be an effective method for delivering neurotrophic factors or antibodies directly into the brain, because a FUS wave causes microbubbles to expand and contract in the capillaries, resulting in the opening of the tight junctions. Such mechanical effects may be responsible for the BBB disruption, and could play an important role in tissue damage due to inertial cavitation. The fact that hemorrhaging follows an FUS-induced BBB disruption, however, indicates that injury to the BBB has occurred and that this technique cannot be considered totally harmless. As such, this safety concern must be carefully considered when employing this method in therapeutic applications to counteract brain disorders.

### SUMMARY OF THE INVENTION

The present invention relates to a method for treating and/or presenting neurodegenerative diseases by using low-intensity pulsed ultrasound, which enhances the neurotrophic factor levels in astrocytes and neurons, memory retention and decreases brain tissue damage by transcranial LIPUS stimulating the subject's brain. The present invention is the first disclosure that LIPUS in the absence of microbubbles can be used to enhance the protein levels of neurotrophic factors in brain cells, improve memory retention and decrease cerebral damage. The results raise the possibility that LIPUS alone could have beneficial effects in treating brain disorders without inducing BBB disruption or causing any tissue damage.

US is a common treatment that has been used in physical therapies for many soft tissue injuries. Previous studies have indicated that appropriate simulation with LIPUS may accelerate the proliferation and differentiation of osteoblasts for the promotion of fracture healing. However, the protein metabolism by which LIPUS alters cell functions remains unclear.

Hence, the present invention describes a method for treating and/or presenting neurodegenerative diseases, including applying a LIPUS apparatus to a subject for increasing the proteins expression of BDNF, GDNF, VEGF, GLUT1, transcription factor c-Fos and CREB in brain astrocyte cells and neurons under low-intensity pulsed ultrasound condition.

For clinical application, exogenous BDNF and GDNF could also have possible side effects such as a pro-epileptic effect and cerebellar damage, respectively. Moreover, AD may be aggravated by a breakdown of the BBB in some patients.

In another aspect, the present invention provides a use of transcranial LIPUS in treating neurodegenerative disorders including, but not limited to, AD, vascular dementia, PD, traumatic brain injury, post-traumatic stress disorder, depression, major depressive disorder, bipolar disorder, stroke, epilepsy, migraine, headache, Huntington's disease, and spinal cord injuries. Since the elevated protein levels of endogenous neurotrophic factors induced by LIPUS lend support to our hypothesis that transcranial LIPUS may be useful for neuroprotection and while not requiring exogenous factors or surgical invasion. The present invention raise the possibility that LIPUS alone could have beneficial effects in treating brain disorders without inducing BBB disruption or causing any tissue damage, and excellent potential for broad applications in neurodegenerative disorders.

In accordance with embodiments of the invention, a medical device for curing neurodegenerative disorders comprises: a LIPUS apparatus comprising a focused piezoelectric transducer, which generates spatial peak temporal average (I_{SPTA}) from 1 mW/cm² to 1 W/cm²; operation frequency from 20K to 16 MHz; a function generator; a power amplifier; and a power sensor module.

In accordance with preferred embodiments of the invention, the LIPUS was generated by a 1-MHz plane piezoelectric transducer. Also, the described LIPUS apparatus is used to treat subject's hemisphere with multiple sonications. The duration of each sonication was 5 min and there was an interval of 5 min between the two sonications. LIPUS stimulation will be bilateral and in others unilateral.

In accordance with embodiments of the invention, brain astrocyte cells and neurons that were exposed to LIPUS exhibited increase in BDNF, GDNF, VEGF, GLUT1, c-Fos and CREB protein expressions. In accordance with another embodiment of the invention, the LIPUS is applied to improve memory retention in memory impairment, and decrease cerebellar damage.

### BRIEF DESCRIPTION OF THE DRAWINGS

FIG. 1 is a schematic diagram of a preferred embodiment that a medical device used to treat neurodegenerative disease.

FIGS. 2A and 2B show the effect of ultrasound parameters on the cell growth. FIG. 2A shows the growth of astrocyte cells sonicated at LIPUS duty cycles ranging from 0 (control) to 100%. FIG. 2B is the time course of the growth from astrocyte cells by treatment with single and multiple LIPUS stimulations at 50% duty cycle. * and # denote significant differences compared to the control at 0 h and the same time points following LIPUS stimulation, respectively. (*, #, p<0.05; **, p<0.01, n=4)

FIG. 3A to FIG. 3D show the increase of BDNF, GDNF, VEGF and GLUT1 protein expressions induced by LIPUS treatment in cultured astrocytes. Rat astrocytes were treated with multiple LIPUS stimulations for a sonication time of 15 min. Cells were subjected to western blot to measure the protein expressions at 0, 2, 4, and 8 h following LIPUS stimulation. * denotes significant differences compared with the treated group at 0 h after LIPUS. (*, p<0.05; **, p<0.01, n=4)

FIG. 4A to FIG. 4C show the related protein expressions of BDNF, GDNF, and VEGF were determined by western blot. Integrin is involved in the LIPUS-induced increase of neurotrophic factor expression. Astrocytes were pretreated with integrin inhibitor (RDG peptide) for 30 min followed by multiple LIPUS stimulations for a sonication time of 15 min. **, p<0.01 as compared with control (n=4).

FIG. 5 shows that concentration dependence of AlCl₃ on cell viability reduction in the absence and presence of LIPUS stimulation. Cells were exposed to various concentrations of AlCl₃ for 24 h in the absence or presence of LIPUS stimulation. ** and # denote significant differences compared to the control cells that received no AlCl₃ and the same concentrations of AlCl₃, respectively. (#, p<0.05; **, p<0.01, n=4)

FIG. 6A to FIG. 6D show the increase of BDNF, GDNF, VEGF and GLUT1 protein expressions due to LIPUS treatment in the rat brain. Each hemisphere was treated with multiple LIPUS stimulations for a sonication time of 15 min. The sonicated regions were subjected western blot to measure the protein expressions at 4 h following LIPUS stimulation. * denotes significant differences in the sonicated hemispheres compared to the ipsilateral control hemispheres. (*, p<0.05; **, p<0.01, n=4)

FIG. 7 shows the effect of ultrasound on memory retention in rats through Morris water maze test. The acquisition latency (AL) on day 20 and retention latency (RL) on days 21 and 42 in AlCl₃-treated rats with or without LIPUS stimulation were observed. *30 and # denote significant differences compared to the individual groups of AL on day 20 and RL on day 21, respectively. † and ‡ denote significant differences between AlCl₃-treated group with and without LIPUS stimulation on days 21 and 42, respectively. (*, #, †, ‡, p<0.05, n=6)

FIG. 8 shows the effect of ultrasound on memory performance in rats through elevated plus maze test. The transfer latency (TL) on days 20, 21, and 42 in AlCl₃-treated rats with or without LIPUS stimulation were observed. * and # denote significant differences compared to the individual groups of TL on days 20 and 21, respectively. † and ‡ denote significant differences between AlCl₃-treated group with and without LIPUS stimulation on days 21 and 42, respectively. (*, #, †, ‡, p<0.05, n=6)

FIG. 9A and FIG. 9B show the effects of LIPUS on aluminum levels and acetylcholinesterase activity respectively in AlCl₃-treated rats. *, #, and † denote significant differences compared to the individual groups of control, LIPUS, and AlCl₃, respectively. (*, #, †, p<0.05, n=4)

FIG. 10 shows the effects of LIPUS on cerebral damage to hippocampus (CAl) and dentate gyrus (DG) in AlCl₃-treated rats. Representative H&E stained brain sections of a control rat, a LIPUS-treated rat, AlCl₃-treated rat, and a LIPUS-treated rat with AlCl₃ administration. LIPUS stimulation on AlCl₃-treated rats had significantly fewer karyopyknosis of cells than AlCl₃-treated rats. The scale bar is 100 μm in amplified regions.

FIG. 11 shows the effects of LIPUS stimulation on apoptotic cell death to hippocampus (CAl) and dentate gyrus (DG) in AlCl3-treated rats. Representative TUNEL stained brain sections of a control rat, a LIPUS-treated rat, AlCl3-treated rat, and a LIPUS-treated rat with AlCl3 administration. LIPUS stimulation on AlCl3-treated rats had significantly fewer apoptotic cells than AlCl3-treated rats. The scale bar is 100 μm in amplified regions.

FIG. 12A and FIG. 12B show the effects of LIPUS stimulation on c-Fos and CREB protein expression. *and # denote significant differences compared to control and the group rats at 4 h following LIPUS stimulation, respectively. (*, #; p<0.05, n=4)

### DETAILED DESCRIPTION OF THE INVENTION

The present invention disclosures a method for treating neurodegenerative disorders by low-intensity pulsed ultrasound. As description below and the corresponding experimental data, the LIPUS transmitted to the cells or rat brain astrocyte cells and neurons increase protein expressions of the endogenous neurotrophic factors and related proteins, and significantly improve memory retention in memory impairment and decrease brain damage.

Without intent to limit the scope of the invention, exemplary instruments, apparatus, methods and their related results according to the embodiments of the present invention are given below. Note that titles or subtitles may be used in the examples for convenience of a reader, which in no way should limit the scope of the invention.

Pulsed Ultrasound Apparatus

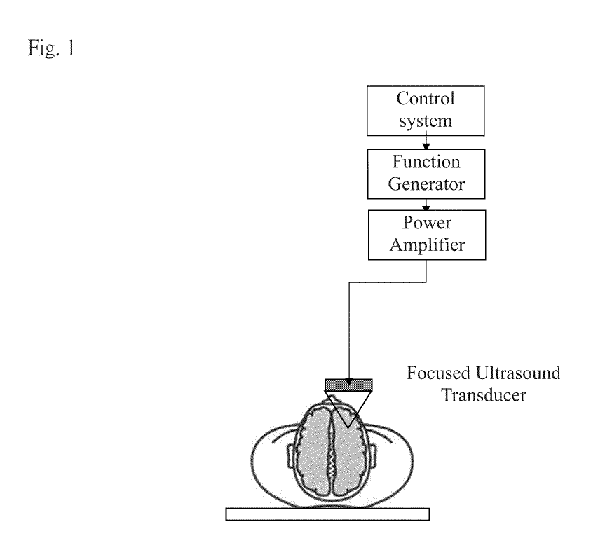

FIG. 1 shows a preferred embodiment that a medical device comprising a focused ultrasound transducer, a function generator, a control system connected to the function generator and a power amplifier, used to treat the hemisphere of a subject.

In in vitro experiments, the LIPUS was generated by a 1-MHz plane piezoelectric transducer (A394S-SU; Panametrics, Waltham, Mass., USA) with 50 ms burst lengths at a 50% duty cycle and a repetition frequency of 10 Hz. In in vivo experiments, the LIPUS was generated by a 1-MHz focused piezoelectric transducer (A392S; Panametrics, Waltham, Mass., USA) with 50 ms burst lengths at a 5% duty cycle and a repetition frequency of 1 Hz. The focused transducer was mounted on a removable cone filled with deionized and degassed water, the tip of which was capped by a polyurethane membrane, with the center of the focal zone placed about 5.0 mm away from the cone tip. The focused transducer was positioned using the stereotaxic apparatus in order to direct the acoustic beam to the desired region (2.3 mm posterior and 2.5 mm lateral to the bregma) of the brain. A function generator (33220A, Agilent Inc., Palo Alto, USA) was connected to a power amplifier (500-009, Advanced Surgical Systems, Tucson, Ariz.) to create the US excitation signal. A power meter/sensor module (Bird 4421, Ohio, USA) was used to measure the input electrical power. The spatial-peak temporal-average intensities (I_{SPTA}) over the plane and focused transducer head were 110 mW/cm² and 528 mW/cm², respectively, and were measured with a radiation force balance (RFB, Precision Acoustics, Dorset, UK) in degassed water. In the in vitro experiments, LIPUS was transmitted from the plane transducer to the bottom of the cell culture plate.

In the in vivo experiments, LIPUS was transmitted from the top of the rat brain. US transmission gel (Pharmaceutical Innovations, Newark, N.J., USA) was used to cover the area between the transducer and the plate or the brain in order to maximize the transmission of the ultrasound. Astrocyte cells and each rat hemisphere were treated by LIPUS with triple sonications. The duration of each sonication was 5 min and there was an interval of 5 min between the two sonications.

Astrocyte Cell Cultures

A RBACs (CTX TNA2) was obtained from the Bioresource Collection and Research Center (BCRC, Hsinchu, Taiwan). The cells were grown on a six-well plate in 95% air-5% CO₂ with Dulbecco's modified Eagle's medium (DMEM; Gibco, New York, USA), which was supplemented with 10% fetal bovine serum (FBS; Biological industries, Kibbutz Beit Haemek, Israel), penicillin (100 U/ml), and streptomycin (100 μg/ml) (Gibco, New York, USA) (pH adjusted to 7.6). Two different cell densities were prepared for subsequent experiments: a cell density of 1×10⁵ cells/well for 3-(4,5-dimethylthiazol-2-yl)-2,5-diphenyltetrazolium bromide (MTT) assays, and a cell density of 1×10⁶ cells/well for western blotting analysis.

Animal Preparation

All procedures were approved according to guidelines stipulated by the Animal Care and Use Committee of National Yang Ming University. Male Sprague-Dawley (SD) rats weighing from 280 to 300 g were used in this study. Before LIPUS stimulation, each animal was anesthetized in the prone position by inhalation of 2% isoflurane in 2 l/min oxygen, and the body temperature was maintained at 37° C. using a heating pad. The rat heads were mounted on a stereotaxic apparatus (Stoelting, Wood Dale, Ill., USA), and the top of the cranium was shaved for LIPUS stimulation. In one experimental protocol, normal rats were used first to evaluate the protein expression of neurotrophic factors 4 h after LIPUS stimulation. In another experimental protocol, the effects of LIPUS on the rats treated with AlCl₃ (100 mg/kg; oral administration) daily for 21 and 42 days were assessed via behavioral test.

Cell Growth Assay

Cell growth was assessed by MTT assay. This method is based on MTT progress to form a corresponding formazan product. After incubation of the cells with 200 μl of 5 mg/ml MTT for 4 h at 37° C. under 95% air-5% CO₂, the cells were then dissolved in 1 ml of DMSO and the absorption was quantified by measuring at 570 nm using a spectrophotometer.

Cell Viability Measurements

LIPUS treatment was started 15 h after the initiation of each cell culture. AlCl₃ (Acros Organics, New Jersey, USA) was dissolved in phosphate buffered saline (PBS) and was made freshly at the beginning of each experiment. The amount of Al was measured from the standard curve prepared with Al standard solution. Various doses (0, 2, 4, 6, and 8 mM) of AlCl₃ were added to RBACs 4 h after LIPUS stimulation, and then cell viability was assessed by MTT assay 24 h after the AlCl₃ treatment.

Western Blotting Analysis

RGD peptide was purchased from Santa Cruz Biotechnology (Paso Robles, Calif.). In in vitro experiments, RBACs were incubated at 0, 2, 4, and 8 hours after multiple LIPUS stimulations. The RBACs were washed in cold PBS and lysed for 30 min on ice with T-Per extraction reagent (Pierce Biotechnology, Inc., Rockford, Ill.). In in vivo experiments, animals were sacrificed 4 h after multiple LIPUS stimulations. Fresh brain tissue in the focal zone was homogenized by T-Per extraction reagent supplemented with the Halt Protease Inhibitor Cocktail (Pierce Biotechnology, Inc.). Lysates were centrifuged and the supernatants were harvested, and protein concentrations were assayed with Protein Assay Reagent (Bio-Rad, California, USA). Samples containing 30 μg protein were resolved on 12% sodium dodecyl sulfate polyacrylamide gel electrophoresis (SDS-PAGE) and transferred to Immun-Blot® polyvinyldifluoride (PVDF) membranes (Bio-Rad, California, USA). After blotting, the membranes were blocked for at least 1 h in blocking buffer (Hycell, Taipei, Taiwan), and then the blots were incubated overnight at 4° C. in a solution with antibodies raised in rabbit against BDNF (1:250, sc-546, Santa Cruz, Calif., USA), GDNF (1:250, sc-328, Santa Cruz, Calif., USA), VEGF (1:250, sc-152, Santa Cruz, Calif., USA), and GLUT1 (1:200, NB110-39113, Novus Biologicals, Colo., USA). After being washed with PBST buffer, the membrane was incubated with the secondary antibodies for 1 h at room temperature. After being washed with PBST buffer, signals were developed using a Western Lightning ECL reagent Pro (Bio-Rad, California, USA). Western blot signals were analyzed and visualized using an ImageQuant⊥ LAS 4000 biomolecular imager (GE Healthcare Bio-Sciences AB, Sweden).

Behavioral Assessment

In an in vivo behavioral experiment, 24 SD rats were randomized into four groups, each with 6 animals. The animals were treated with vehicle and served as the control group. In the second group, animals received LIPUS treatment for 49 days. Animals in the third group served as the AlCl₃-group and only received AlCl₃ (100 mg/kg) daily for 42 days. Along with LIPUS pretreatment for 49 days, the animals in the fourth group were challenged with AlCl₃ (100 mg/kg) daily for the last 42 days to induce learning deficits and amnesia. The acquisition and retention of a spatial navigation task was assessed by Morris water maze. The pool is a custom-made black (200 cm×60 cm) filled with water (23±2° C.). Opaque curtains surrounded the maze and were affixed with high-contrast visual cues (an X, a triangle, a circle, and a square). The pool was divided arbitrarily into four equally-sized quadrants (called zones I, II, III, and IV). The escape platform is a custom-made, clear plastic stand with a circular top measuring 20 cm in diameter.

It sits approximately 2 cm above the surface of the water during the acquisition phase. The rats received a training session consisting of four trials on day 20 from the start of AlCl₃ administration. The start locations were varied from trial to trial, with the rats being gently placed in the water facing towards the wall of the pool. The maximum swim time for the acquisition trial was 90 s, after which the rat was guided to the platform and remained there for 20 s following escape. The time spent by the rat to reach the platform was recorded and termed as AL. After completing the training trial, the rats were returned to the home cages and a 5 min gap was timed between the subsequent trials. Then, a similar platform was placed in the pool 2 cm below the water level for the maze retention phase. One day after the AL was recorded, the given rat was placed randomly at one of the edges facing the wall of the pool and tested for retention of response. The times spent to reach the platform on days 21 and 42 following the start of AlCl₃ administration were measured and expressed as RL. The elevated plus maze consisted of two open arms (50 cm×12 cm), crossed with two closed walls raised 66 cm from floor level. Each rat was placed at one end of the open arm facing away from the center portion of the maze. The time spent by the rat to move from the open arm to the closed arm was measured as the TL on day 20 from the start of AlCl₃ administration. The rats remaining in the open arm without entering into the closed arm within 90 s were pushed on the back into one of the enclosed arm and TL was recorded as 90 s. Similarly, retention of memory was evaluated as TL on days 21 and 42.

Effect of Ultrasound on Cell Growth

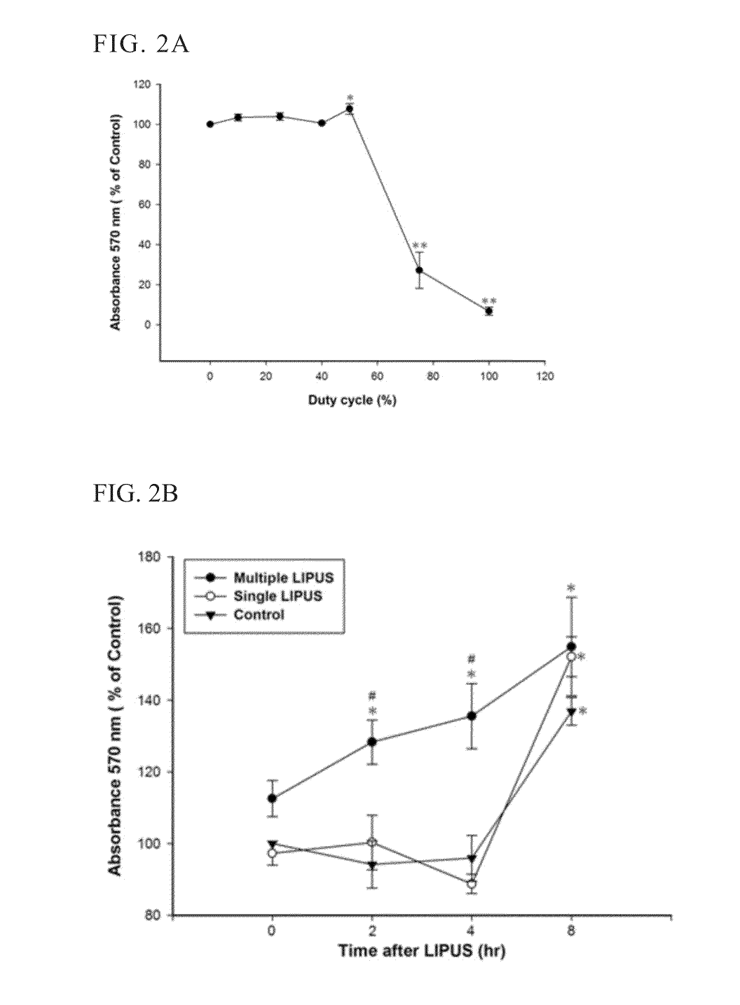

The effect of LIPUS stimulation on the cell growth in astrocytes was evaluated (FIG. 2). The astrocytes that were subjected to LIPUS demonstrated an increase in cell growth at 50% duty cycle (FIG. 2A). At duty cycle values higher than 50% for a single sonication, the cell growth was rapidly decreased as a function of the duty cycle. The increase of cell growth was significantly higher with multiple sonications than with a single sonication within 8 h after LIPUS stimulation (FIG. 2B). The protein expressions of the neurotrophic factors were therefore quantified in astrocytes following multiple sonications with 50% duty cycle for a sonication time of 15 min.

Ultrasound Enhanced the Expressions of BDNF, GDNF, VEGF, and GLUT1 in Astrocytes

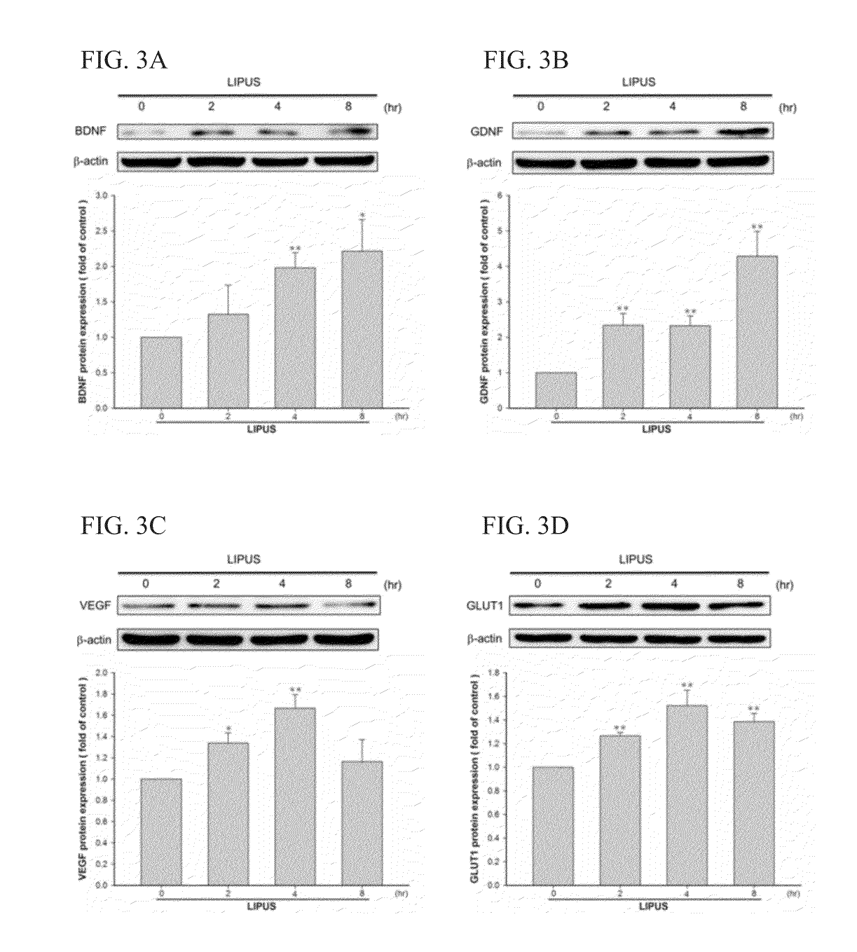

Rat brain astrocyte cells (RBACs) that were exposed to LIPUS exhibited a time-dependent increase in BDNF, GDNF, VEGF, and GLUT1 protein expressions (FIG. 3). The values reached maximums for BDNF and GDNF at 8 h after LIPUS stimulation (FIGS. 3A and B). On the other hand, the profile of protein expression for VEGF was similar to that for GLUT1, with peak values for both occurring at 4 h (FIGS. 3C and D).

Ultrasound Increased Neurotrophic Factor Expression via Integrin

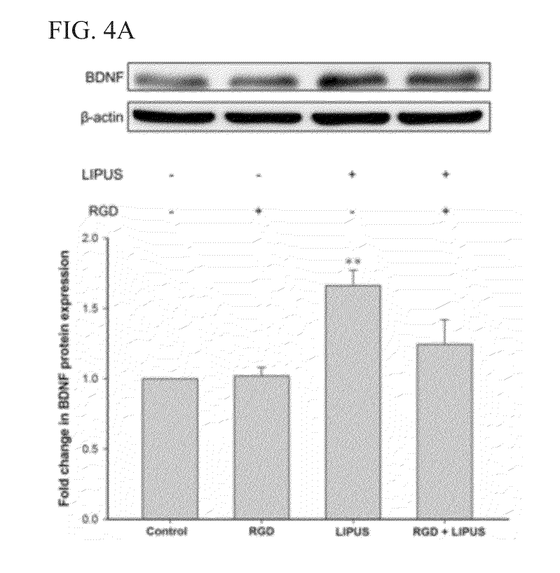

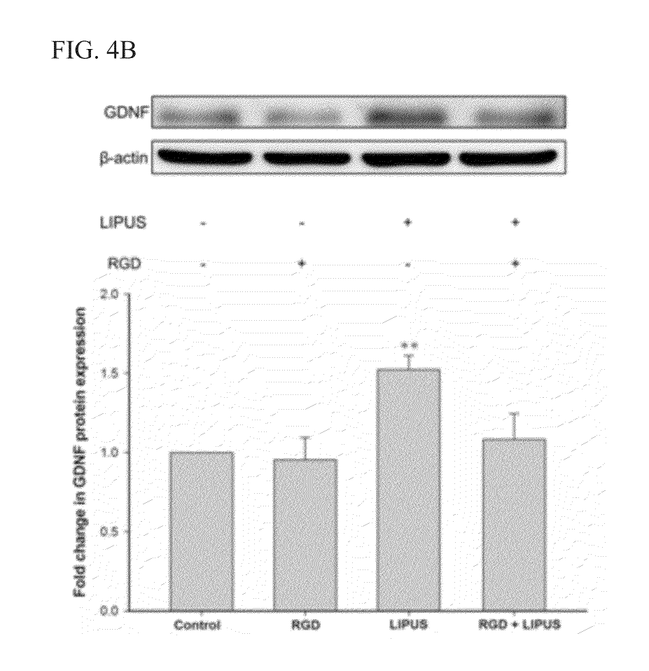

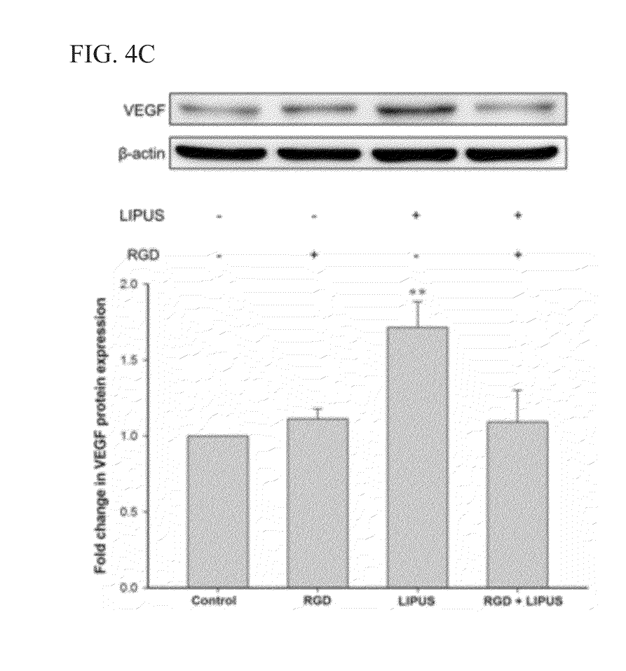

It has been demonstrated that transient LIPUS stimulation increases the expression of integrins in cell membranes (Yang, et al., Bone, 36: 276-283, 2005). Some studies have suggested that integrins may act as LIPUS-sensitive receptors and involve the activation of several protein kinases in the downstream signaling pathway (Hsu, et al., Cellular signalling, 19: 2317-2328, 2007). Here, we examined the effect of disintegrin RGD peptide on the LIPUS-induced increase of protein expressions for BDNF, GDNF, and VEGF, and found that pretreatment of cells for 30 min with RGD peptide markedly inhibited the LIPUS-induced increase of those proteins (FIGS. 4A-C). These data suggest that LIPUS-induced neurotrophic factor expression may occur via activation of integrin receptor signaling.

Effect of Ultrasound on Cell Viability

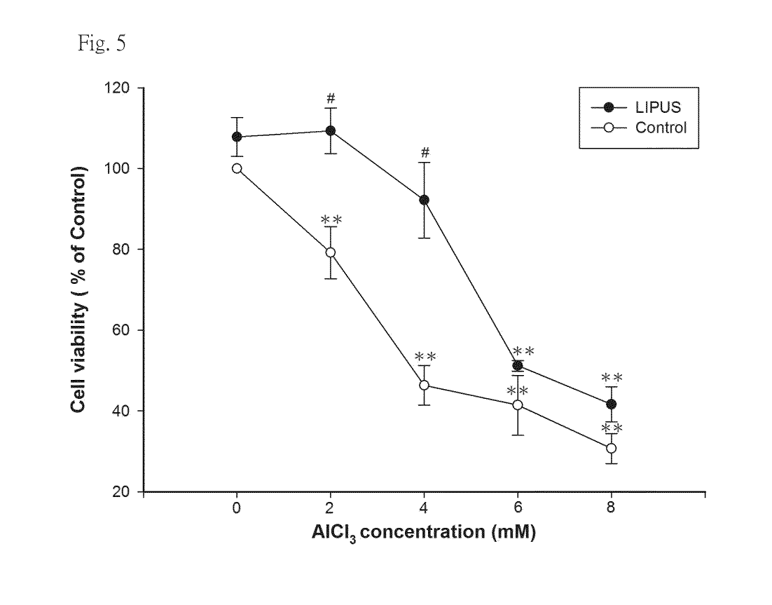

The cytotoxicity of aluminum chloride (AlCl₃) for astrocyte cells was determined by a decrease in the tetrazolium (MTT) activity (FIG. 5). The cells were treated with various concentrations of AlCl₃ (0-8 mM) in the absence or presence of multiple LIPUS stimulations. In the control group, the dose-response curve for aluminum toxicity was steep. In the experimental group, the median lethal dose was shifted from 3.77 to 6.25 mM AlCl₃ by multiple LIPUS stimulations. The protective effect of LIPUS against AlCl₃-induced cell degeneration was significantly observed in the MTT activity of the cells at the lower doses of AlCl3 (2 and 4 mM). There was also a modest increase (10-12%) in cell viability at the higher doses of AlCl₃ (6 and 8 mM) in LIPUS-treated cells, but this was not statistically significant.

Effect of Ultrasound on Protein Expression of BDNF, GDNF, VEGF, and GLUT1 in Rat Brain

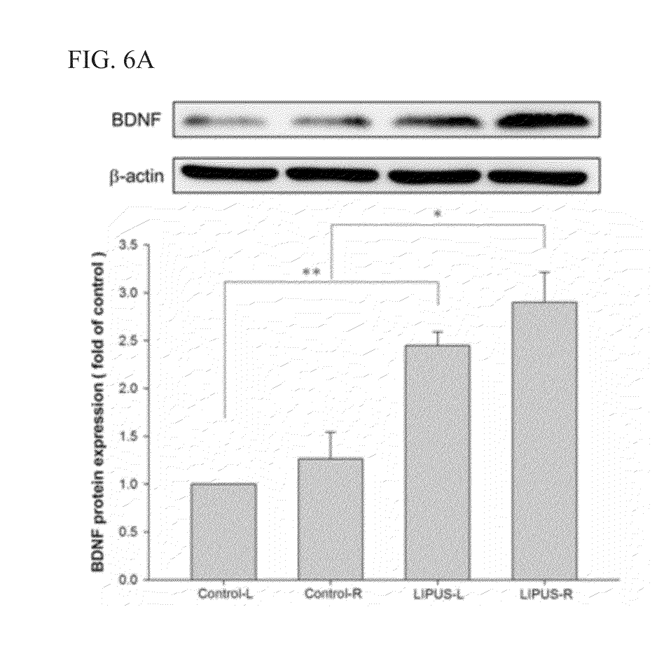

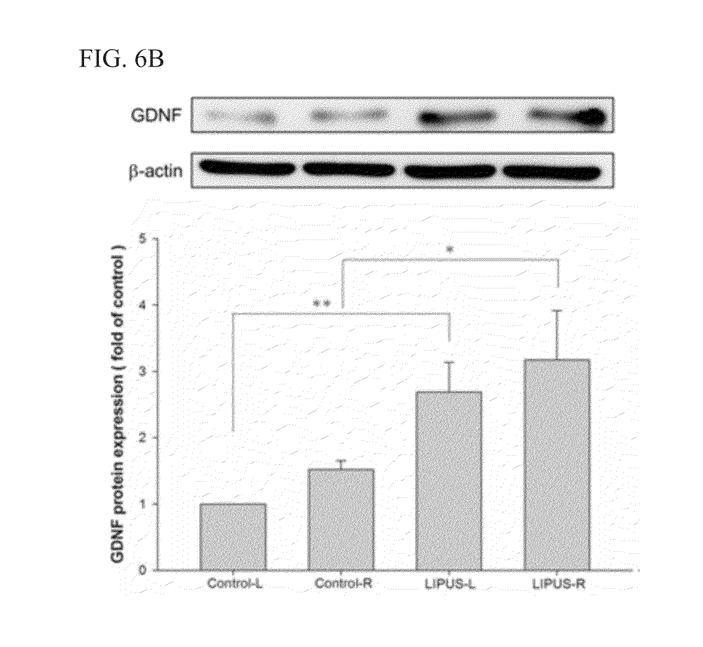

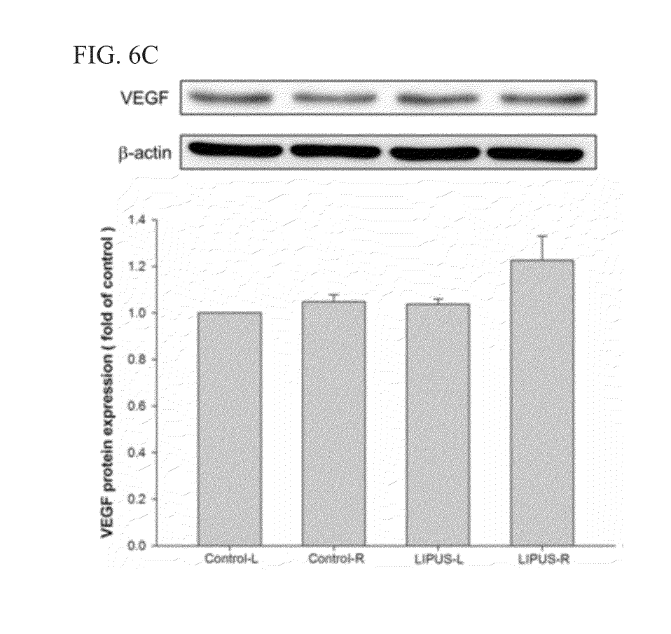

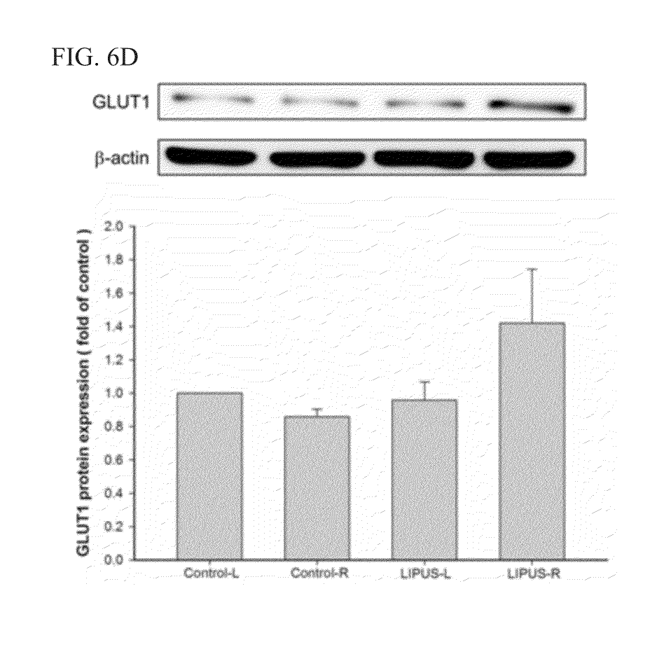

To further confirm the effect of LIPUS on the protein levels of neurotrophic factors in the brain, bilateral rat hemispheres were exposed to multiple LIPUS stimulations for a sonication time of 15 min. Western blot analysis was used to examine the endogenous protein expressions 4 h after LIPUS stimulation. Whether LIPUS stimulation was applied to the right or left hemisphere, the protein expressions of BDNF and GDNF in the stimulated hemisphere were significantly enhanced compared with the same expressions in the ipsilateral control hemisphere (FIG. 6A and FIG. 6B). However, no significant differences were found for the protein expressions of VEGF and GLUT1 in the sonicated hemisphere as compared with the ipsilateral control hemisphere (FIGS. 6C and D).

Effect of Ultrasound on Memory Performance in Aluminum Chloride-treated Rats

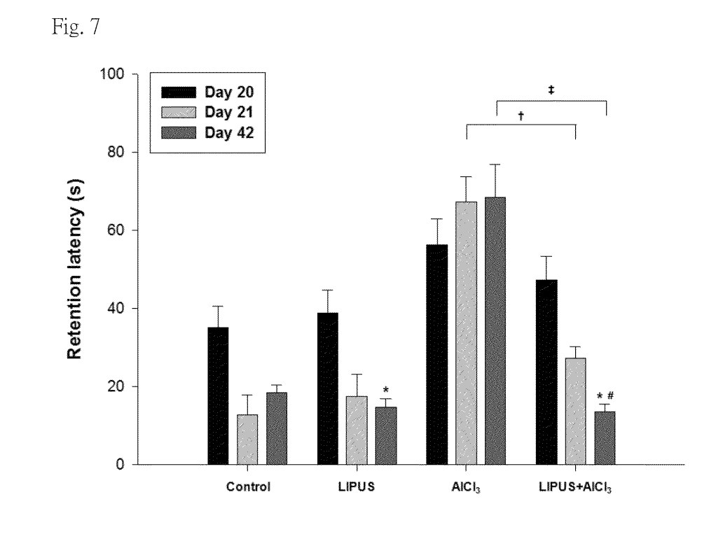

Rats treated only with AlCl₃ showed learning and memory deficits in the Morris water maze task compared to control group rats (FIG. 7). There was a significant increase in the mean acquisition latency (AL) of the AlCl₃-treated group when compared to the control group on day 20. By contrast, a combination treatment of LIPUS and AlCl₃ resulted in a mildly decreased AL as compared to rats treated only with AlCl₃ on day 20. Following training, the mean retention latency (RL) was significantly decreased in the control group on days 21 and 42, respectively, as compared to the AL on day 20. The LIPUS treatment of AlCl₃-treated rats resulted in a significant decline in RL on days 21 and 42, respectively, as compared to the RL in rats treated only with AlCl₃. These results suggest that the retention performance for the spatial navigation task was improved by LIPUS stimulation.

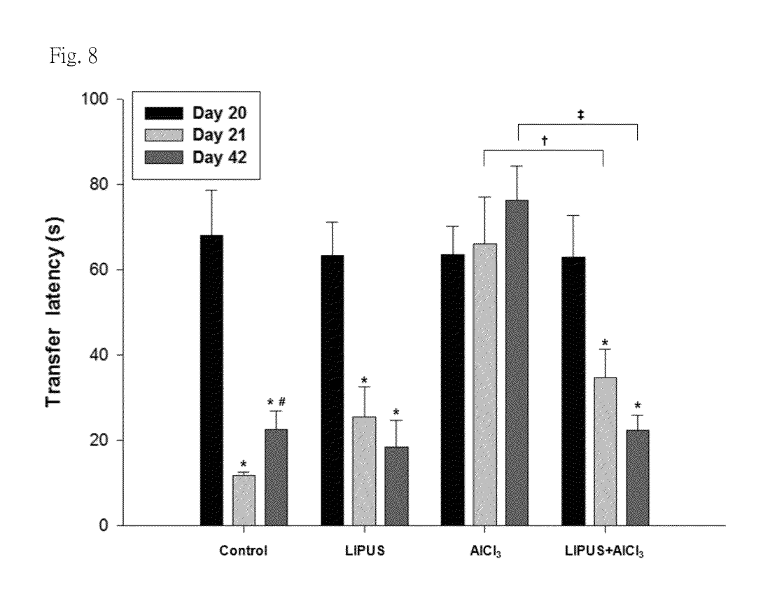

In the elevated plus maze, memory was evaluated and termed as transfer latency (TL). On day 20, mean TL for each group was relatively stable and showed no significant difference (FIG. 8). Following training, mean TL in control rats on days and 42 were significantly decreased as compared to TL on day 20, respectively. In contrast, no significant differences were found in the mean TL of AlCl₃ treated group on days 21 and 42 as compared to pre-training TL on day 20. There was a significant increase in the mean TL of AlCl₃ treated group when compared to control group on days 21 and 42. However, there was no statistical change in the combination of LIPUS and AlCl₃ offered treated group as compared to control group on days 21 and 42. Furthermore, the LIPUS treatment of AlCl₃-treated rats resulted in a significant decline in TL on days 21 and 42, respectively, as compared to the TL in rats treated only with AlCl₃. The LIPUS stimulation alleviated the AlCl₃-induced learning and memory deficits in rats.

Estimation of Aluminum Concentration and Acetylcholinesterase Activity

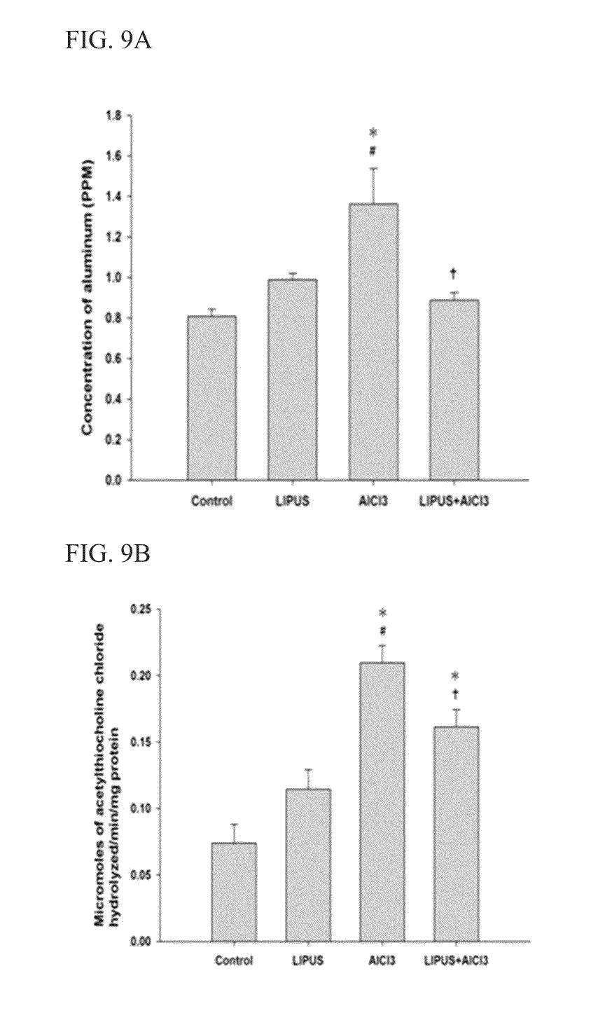

AlCl₃-treated rats showed significant increase in the aluminum concentration and AChE activity as compared to control. Chronic LIPUS stimulations in AlCl₃-treated rats significantly attenuated the increase in aluminum concentration and AChE activity as compared to the AlCl₃-treated rats (FIG. 9). However, no significant differences were found in aluminum concentration and AChE activity after LIPUS stimulation in normal rats as compared to control.

Histological Observation

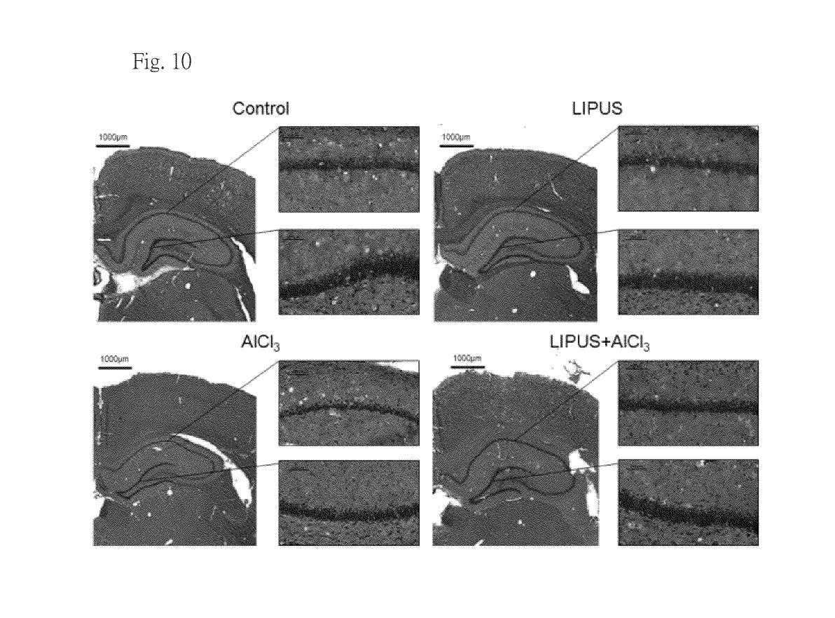

As shown in FIG. 10, a karyopyknosis was observed in the hippocampal CAl and dentate gyms (DG) of AlCl₃-treated rats with or without LIPUS stimulation. Furthermore, fewer karyopyknosis of cells were found in AlCl3-treated rats with LIPUS stimulation compared with the AlCl₃ group. The LIPUS treatment ameliorates the cerebral damage in the AlCl₃-treated rats.

Effects of LIPUS Treatment on Apoptotic Cell Death

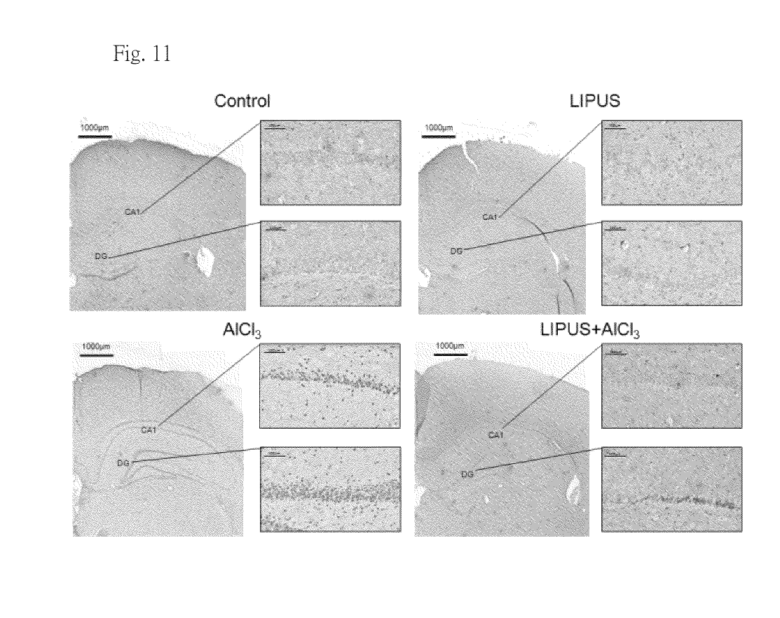

As shown in FIG. 11, TUNEL-positive cells were observed in the hippocampal CAl and dentate gyms (DG) of AlCl₃-treated rats with or without LIPUS stimulation. Furthermore, fewer apoptotic cells were found in AlCl₃-treated rats treated with LIPUS compared with the AlCl₃ group. No apoptotic cells were found in the normal brain after LIPUS stimulation.

Ultrasound Increased Neurotrophic Factors, c-Fos and Transcription Factor CREB Expression

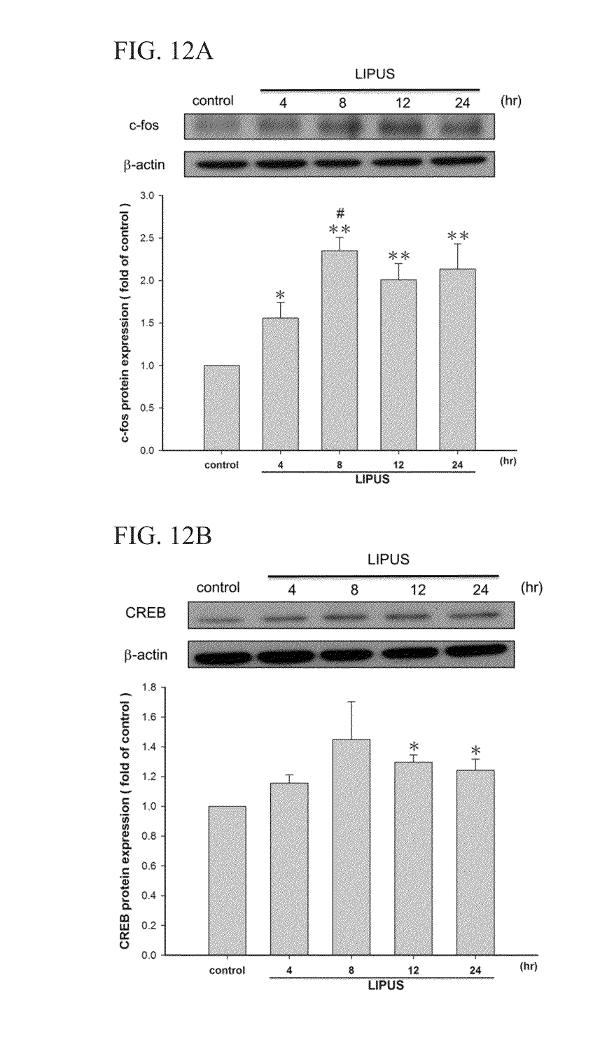

Another preferred embodiment as shown in FIG. 12. Compared with control group, the rats exposed to LIPUS exhibited a time-dependent increase in c-Fos and CREB protein expressions. The values reached maximums for c-Fos and CREB at 8 h after LIPUS stimulation (FIG. 12A). CREB is a central mediator of neurotrophic factor regulation and responses. Neurogenesis is involved in spatial learning and memory and is regulated by the neurotrophic factor c-Fos. This present invention discloses that LIPUS stimulation is applied in treating and/or presenting neurodegenerative diseases via neurotrophin-regulated signalling pathways.

What is claimed is:

1. A method for stimulating neurotrophic factor expression in brain cells, comprising:
providing a pulsed ultrasound apparatus;
placing the pulsed ultrasound apparatus on a subject; and
generating a low-intensity pulsed ultrasound (LIPUS) to targeted cells of a subject by the pulsed ultrasound apparatus with a spatial peak temporal average (I_{SPTA}) from 1 mW/cm² to 1 W/cm² and an operation frequency ranging from 20K to 16 MHz;
wherein the expression level of neurotrophic factor in the targeted cells is increased in comparison to non-targeted cells.

2. The method of claim 1, wherein the pulsed ultrasound apparatus comprises:
a focused piezoelectric transducer, which generates spatial peak temporal average (I_{SPTA}) from 1 mW/cm² to 1 W/cm²; operation frequency ranging from 20K to 5 MHz,
a function generator,
a power amplifier connected to the function generator and,
a power sensor module.

3. The method of claim 1, wherein the operation frequency is 1 MHz.

4. The method of claim 2, wherein the operation frequency is 1 MHz.

5. The method of claim 1, wherein the subject is human.

6. The method of claim 1, wherein the targeted cells are astrocyte cells or neurons.

7. The method of claim 1, wherein the neurotrophic factor comprises BDNF, GDNF, VEGF, and c-Fos proteins.

8. The method of claim 1, wherein the method is used to treat and/or prevent a neurodegenerative disease.

9. The method of claim 7, wherein the neurodegenerative disease comprising Alzheimer's disease (AD), vascular dementia, Parkinson's disease (PD), traumatic brain injury, post-traumatic stress disorder, major depressive disorder, bipolar disorder, stroke, epilepsy, migraine, headache, Huntington's disease, and spinal cord injuries.

10. The method of claim 1, wherein the low-intensity pulsed ultrasound is further combined with one or more treatment methods selected from the group consisting of transcranical magnetic stimulation (TMS), transcranial direct current stimulation (tDCS), deep-brain stimulation (DBS).

11. The method of claim 1, wherein the method is used to protect cells against aluminum-induced neurotoxicity.
